(19) United States
(12) Patent Application Publication
(10) Pub. No.: US 2021/0008090 A1
(43) Pub. Date: Jan. 14, 2021

(54) PARTICULATE SUBSTANCE MIXTURE, PREFERABLY FOR USE IN THE PROPHYLAXIS AND/OR TREATMENT OF A RESPIRATORY DISORDER

(71) Applicants: Bart Zonnenberg, Nieuwegein (NL); Constantin Adams, Essen (DE); Anke Ingeborg Riethmüller, Tübingen (DE)

(72) Inventors: Bart Zonnenberg, Nieuwegein (NL); Constantin Adams, Essen (DE); Joachim Riethmüller, Tübingen (DE)

(21) Appl. No.: 16/336,655

(22) PCT Filed: Sep. 27, 2017
(86) PCT No.: PCT/EP2017/074513

§ 371 Date: Mar. 26, 2019

(30) Foreign Application Priority Data
Sep. 27, 2016 (DE) 10 2016 218 604.3

(51) Int. Cl. A61K 31/7036; A61K 9/16; A61K 33/06; A61K 33/02; A61K 33/14; A61K 47/26; A61K 9/00; A61K 31/4375; A61K 31/198; A61K 31/137; A61P 11/00

(52) U.S. Cl. CPC A61K 31/7036; A61K 9/1611; A61K 33/06; A61K 33/02; A61K 33/14; A61P 11/00; A61K 9/0053; A61K 31/4375; A61K 31/198; A61K 31/137; A61K 47/26

(57) ABSTRACT

A particulate substance mixture, preferably for use in the prophylaxis and/or treatment of a respiratory disorder includes a mucolytic substance and amorphous silicon dioxide. The mixture may also have the following characteristics: the amorphous silicon dioxide is hydrophilic silicon dioxide, the amorphous silicon dioxide has a mean particle diameter ≤20 μm, and an amount of the amorphous silicon dioxide is 0.1% by weight to 10% by weight, based on the total weight of the particulate substance mixture.

### TECHNICAL FIELD

This disclosure relates to a particulate substance mixture, preferably for use in the prophylaxis and/or treatment of a respiratory disorder, a medicinal product, preferably for use in the prophylaxis and/or treatment of a respiratory disorder, a dosing unit, a device that administers the particulate substance mixture, the medicinal product or the dosing unit, and a method of producing the particulate substance mixture or medicinal product.

### BACKGROUND

One major challenge facing modern medicine and pharmacology is the treatment of diseases of the respiratory system. This applies above all to chronic pulmonary disease such as, e.g., mucoviscidosis, also known as cystic fibrosis (CF), primary ciliary dyskinesia (PCD), chronic obstructive pulmonary disease (COPD) and bronchial asthma.

It is characteristic of the above-mentioned diseases that patients frequently suffer from a sharply reduced capacity to liquefy and cough up bronchial secretions. The viscous mucus in the bronchi can lead to chronic cough, bronchiectasis, frequently recurring pulmonary infections and severe pneumonia.

The treatment of respiratory diseases includes numerous approaches such as, e.g., administration of secretolytics, antibiotics, fat-soluble vitamins and the like, as well as antiinflammatory therapy.

For example, one approach is administration of so-called Pulmozym®. This is a recombinant human deoxyribonuclease, i.e., an enzyme that cleaves extracellular DNA. This allows the bronchial mucus to be liquefied and better dissolved. A drawback is that this form of treatment is relatively expensive. This drawback is compounded by the fact that the therapeutic approach is relatively complex, requiring a special inhalation device. Moreover, several inhalations over the course of a day are required to provide patients with a sufficient dose of Pulmozym®. This not only makes treatment of such patients time-consuming, but also limits their mobility.

An alternative therapeutic approach is wet inhalation of hypertonic saline solution. There have been isolated reports of side effects of that treatment, primarily in the form of pulmonary obstruction. Because of the requirement for a special inhalation device, that form of treatment is also relatively complex and time-consuming. For example, the required duration of inhalation, depending on the dose to be inhaled, is ordinarily 10 to 20 minutes per day.

A further approach to the treatment of respiratory diseases is so-called “halotherapy.” Halotherapy is carried out in a climatically suitable chamber or salt cave. This is a special room in which small amounts of micronized table salt are released into the ambient air. For treatment, the patients remain in the chamber for 30 to 60 minutes and inhale the salt released into the ambient air.

Halotherapy generally allows an improvement in pulmonary function parameters and the frequency of exacerbations to be achieved. The results of clinical studies conducted on a mixed collective of patients with asthma, COPD, bronchiectasis and CF showed a positive action on pulmonary function and an improvement in disease symptoms. In particular, positive effects were achieved after a two-week daily treatment phase with 45-minute inhalation (cf. Hedman et al. (2006), The Effect of Salt Chamber Treatment on Bronchial Hyperresponsiveness in Asthmatics, Allergy 61, pp. 605-610; Chervinskaya and Zilber (1995), Halotherapy for Treatment of Respiratory Diseases, Journal of Aerosol Medicine 3, pp. 221-232; Chervinskaya (2003), Halotherapy of Respiratory Diseases, Physiotherapy, Balneology and Rehabilitation 6, pp. 8-15).

In cystic fibrosis or mucoviscidosis, it was found in a small pilot study that halotherapy had a positive effect on pulmonary secretolysis and pulmonary function parameters.

However, a drawback of halotherapy is the fact that it is ordinarily extremely complex for the patients using it. For example, a session in the air-conditioned chamber ordinarily lasts about 45 minutes and must be regularly repeated over a period of several days or every day. This is compounded by the fact that air-conditioned chambers are not available to most patients.

The principle of halotherapy is increasingly being adopted in the so-called wellness field. Treatment of the respiratory tract is frequently carried out via wet inhalation with hypertonic saline solution. This causes an improvement in pulmonary function, and there are also indications that the improved bronchial hygiene resulting from that treatment reduces chronic inflammation and the frequency of exacerbation of infections. Patients benefit from the osmotic action of the hypertonic saline solution, which increases the water content of the bronchial mucus produced and results in improved pulmonary clearance and secretolysis (cf. Ratjen (2006), Restoring Airway Surface Liquid in Cystic Fibrosis, New England Journal of Medicine 354, pp. 291-293).

WO 01/62264 A2 discloses a modified form of halotherapy for treatment of common colds and influenza in which patients are administered sodium chloride as a dry powder by a dispenser, wherein the sodium chloride is in the form of an aerosol and the aerosol particles have a mean particle diameter of 2 μm to 6 μm.

However, the principle of the method known from WO 01/62264 A2 is disadvantageous in that chronic pulmonary diseases that are significantly more critical from a medical standpoint are not taken into consideration. Moreover, the use of a dry powder involves the risk that the particles may agglomerate or clump into larger aggregates, which in extreme cases can in particular cause deep pulmonary deposition, with the result that treatment of chronic pulmonary diseases is no longer possible.

It could therefore be helpful to provide improved treatment, preferably of respiratory disorders or diseases, that at least largely avoids the drawbacks of known treatments.

### SUMMARY

We provide a particulate substance mixture for use in the prophylaxis and/or treatment of a respiratory disorder, including a mucolytic substance and amorphous silicon dioxide.

We also provide a medicinal product for use in the prophylaxis and/or treatment of a respiratory disorder, including a particulate substance mixture for use in the prophylaxis and/or treatment of a respiratory disorder, including a mucolytic substance and amorphous silicon dioxide.

We further provide a dosing unit including a particulate substance mixture for use in the prophylaxis and/or treatment of a respiratory disorder, including a mucolytic substance and amorphous silicon dioxide.

We still further provide a method of producing the particulate substance mixture for use in the prophylaxis and/or treatment of a respiratory disorder, including a mucolytic substance and amorphous silicon dioxide, including a) mixing a mucolytic substance and amorphous silicon dioxide, and b) grinding, atomizing or micronizing the resulting particulate substance mixture.

### DETAILED DESCRIPTION

We provide a particulate, preferably powdered substance mixture, preferably for use in the prophylaxis and/or treatment of a respiratory disorder or disease.

The particulate substance mixture is characterized in particular by comprising a mucolytic substance and amorphous, i.e., non-crystalline, silicon dioxide.

We found that agglomeration or clumping of mucolytic substance particles in the presence of amorphous silicon dioxide particles as a separating agent can be prevented, hindered or delayed, at least for a particular period of time, for example, a period of 1 to 2 years. This makes it possible to at least significantly reduce the risk of treatment failure resulting from particle agglomeration or clumping.

A further advantage lies in significantly improved long-term stability and shelf life.

We also surprisingly found that administration of amorphous silicon dioxide particles did not cause any harmful adverse effects in the patients and, astonishingly, the particles were found to be highly biocompatible.

A further advantage is that the particulate configuration of the substance mixture makes it possible in dry inhalational administration to achieve extremely high treatment concentrations, in particular of the mucolytic substance, in the ambient air. For example, the treatment concentrations can be 2 mg/m³ to 15 mg/m³ of ambient air. To obtain corresponding concentrations, for example, in nasal inhalation with hypertonic saline solution, it would be necessary to administer repeated administrations of approx. 5 ml of hypertonic saline solution throughout the day using an inhalation nebulizer.

This provides the further advantage of making it possible to significantly shorten the period of treatment with the substance mixture, in particular to a few seconds, in particular when the substance mixture is administered by capsule inhalation, preferably cumulative capsule inhalation. This makes the substance mixture particularly well-suited for multiple administrations per day, but without requiring the patients to spend an undue period of time. This in turn allows the patients to be treated without any significant impairment of quality of life.

The term “particulate substance mixture” means a substance mixture in the form of particles, preferably in the form of powder particles.

The term “respiratory disorder” means a disorder or disease of the respiratory tract or a portion thereof. The respiratory tract can refer to the respiratory tract of a human or a non-human mammal. It is preferably the respiratory tract of a human patient. In other words, the term “respiratory disorder” preferably refers to a disorder or disease of the respiratory tract or a portion thereof in a human.

The respiratory tract refers to all portions of the respiratory system that serve as conduction paths between the external environment and the alveoli. The respiratory tract can be divided into the upper respiratory tract and the lower respiratory tract. The upper respiratory tract includes the nasal cavity and paranasal sinuses, the oral cavity, and the throat (pharynx). The lower respiratory tract includes the voice box (larynx), the windpipe (trachea), the bronchi, the bronchioles such as the lobular bronchioles (bronchioli lobulares), the terminal bronchioles (bronchioli terminales), the respiratory bronchioles (bronchioli respiratorii), the alveolar ducts and the alveoli.

The term “mucolytic substance” means a substance that promotes or favors dissolution or detachment of mucus, in particular bronchial or nasal mucus, preferably bronchial mucus, for example, by expectoration, coughing, sneezing and/or blowing one's nose. The term “mucolytic substance” thus means in particular secretolytics, mucolytics and/or secretomotorics. Reference is made to the explanations given below with respect to suitable mucolytic substances.

The term “secretolytic” or “secretolytically active substance” means a substance that stimulates the production of thin mucus, in particular bronchial or nasal mucus, and preferably bronchial mucus.

The term “mucolytic” or “mucolytically active substance” means a substance that liquefies existing mucus, in particular existing bronchial or nasal mucus, and preferably existing bronchial mucus.

The term “secretomotoric” or “secretomotorically active substance” means a substance that stimulates motility of the cilia, in particular the nasal mucosa or bronchi, preferably the bronchi, and thus allows improved removal of mucus, in particular bronchial or nasal mucus, and preferably nasal mucus.

The term “mucolytic substance” can further refer to one mucolytic substance (singular) or a plurality, in particular a mixture, of different mucolytic substances (plural).

The term “pyrogenic silicon dioxide” means a preferably amorphous and in particular non-porous silicon dioxide powder having a mean particle diameter of 5 nm to 50 nm, a specific area of 50 m²/g to 600 m²/g, in particular 50 m²/g to 400 m²/g, and a density of 30 kg/m³ to 250 kg/m³, in particular 160 kg/m³ to 190 kg/m³. Such a silicon dioxide powder is ordinarily produced by flame hydrolysis. For this purpose, sand is used as a starting material. The sand is first reduced with carbon, and the resulting silicon is then reacted with chlorine to form silicon tetrachloride. The final step is high-temperature pyrolysis of the silicon tetrachloride with oxyhydrogen gas. In this process, a homogeneous mixture of vaporous silicon tetrachloride, hydrogen, oxygen and an inert gas is incinerated with a burner in a cooled combustion chamber. Hydrochloric acid is formed as a byproduct. In the flame, droplike silicon dioxide particles are first formed, and then settle in a chainlike manner adjacent to one another and thus form three-dimensional secondary particles by branching. This gives rise to a white powder having an extremely low (bulk) density and a high surface area.

The term “precipitated silicon dioxide” means an amorphous silicon dioxide formed chiefly by precipitation processes with water glass as a starting material. The water glass can be obtained by decomposing quartz sand with sodium carbonate or potassium carbonate.

The term “mean particle diameter” means the following.

The point of departure in the following explanations is the fact that as a rule, the particles of a substance or substance mixture do not have a constant uniform particle diameter, even after comminution steps have been previously carried out. Rather, there is a particle size distribution between larger and smaller particles. If this particle size distribution is measured, this ordinarily yields distribution curves that have the distribution of the particles around a “mean particle size.” In this example, there is frequently a monomodal distribution of the particles around a (single) mean value. However, multimodal distributions with at least two different particle size distributions are also possible.

The graphically depictable distribution curve can also be represented based on diameter values. The so-called D₅₀ value indicates the mean particle size. The D₅₀ value means that 50% of the particles are smaller than the indicated value. Unless otherwise indicated, the term “mean particle diameter” corresponds to this D₅₀ value. A mean particle diameter <5 μm thus means that 50% of the corresponding particles have a diameter <5 μm.

Particle size distributions can be determined in various ways, wherein particle analysis by laser diffraction is particularly well-suited for the particle sizes specified herein. To our knowledge, laser diffraction (laser diffractometry) is the most widely-used method of analyzing particle size distributions. It can be used over a relatively broad range of particle sizes (0.1 μm to 3,000 μm), wherein automated, commercially available devices can be used for this analysis. Particle analysis by laser diffraction is described in ISO 13320-1, the content of which is expressly incorporated herein by reference. A commercially available device for this laser diffractometry is the scattered light measuring unit LS 13320 from Beckman Coulter, California, USA. The mean particle diameter of the substances used the examples below was also measured using this device.

The term “powder” means a particulate substance or a particulate substance mixture whose particles have a mean particle diameter ≤20 μm, in particular 0.1 μm to 18 μm, preferably ≤10 μm, more preferably 0.1 μm to 6 μm, and particularly preferably 0.1 μm to 5 μm. Preferably, the term “powder” means a particulate substance or a particulate substance mixture whose particles have a mean particle diameter <5 μm, in particular 0.1 μm to 4 μm, preferably 0.1 μm to 3 μm, more preferably 0.1 μm to 2 μm, and particularly preferably 0.1 μm to 1 μm.

The term “dry powder” means an anhydrous or essentially anhydrous powder. Accordingly, the term “dry salt” means an anhydrous or essentially anhydrous salt, and preferably an anhydrous or essentially anhydrous inorganic salt.

The term “essentially anhydrous powder” preferably means a powder having a water content ≤0.4% by weight, in particular ≤0.3% by weight, preferably ≤0.2% by weight, and particularly preferably ≤0.1% by weight based on the total weight of the powder. Accordingly, the term “essentially anhydrous salt” preferably means a salt having a water content ≤0.4% by weight, in particular ≤0.3% by weight, preferably ≤0.2% by weight, and particularly preferably ≤0.1% by weight based on the total weight of the salt.

The term “dry inhalation” means administration by inhalation of a dry powder, in particular a dry salt.

The term “micronizing” means a process or method of significantly comminuting particles by grinding or atomizing. In particular, the term “micronizing” means a grinding or atomizing method by which particulate substances or particulate substance mixtures with a mean particle diameter ≤20 μm, in particular 0.1 μm to 18 μm, preferably ≤10 μm, more preferably 0.1 μm to 6 μm, and particularly preferably 0.1 μm to 5 μm can be produced. Preferably, the term “micronizing” means a grinding or atomizing method by which particulate substances or particulate substance mixtures with a mean particle diameter <5 μm, in particular 0.1 μm to 4 μm, preferably 0.1 μm to 3 μm, more preferably 0.1 μm to 2 μm, and particularly preferably 0.1 μm to 1 μm can be produced.

The term “capsule inhalation” means inhalation or administration by inhalation of the contents of a capsule, in particular the particulate, preferably powdered contents of a capsule.

Preferably, the amorphous silicon dioxide is a synthetic silicon dioxide. For example, the amorphous silicon dioxide can be pyrogenic silicon dioxide, preferably silicon dioxide produced by flame hydrolysis and then ground or produced by flame hydrolysis and then micronized. In particular, the amorphous silicon dioxide can be silicon dioxide produced by flame hydrolysis and then micronized. Alternatively, the amorphous silicon dioxide can be precipitated silicon dioxide, preferably silicon dioxide precipitated and then ground or precipitated and then micronized. In particular, the amorphous silicon dioxide can be silicon dioxide that is precipitated and then micronized.

It is particularly preferred for the amorphous silicon dioxide to be hydrophilic, i.e., non-hydrophobic, silicon dioxide. This is advantageous in that, because of its hydrophilic nature, the amorphous silicon dioxide can bind any traces of moisture or water present in the particulate substance mixture, thus making it possible to prevent, or at least significantly delay, retrograde water absorption that promotes particle agglomeration or clumping by the mucolytic substance. This in turn improves, and in particular prolongs, the therapeutic efficacy of the particulate substance mixture, for example, by ensuring that the substance mixture, in particular the mucolytic substance and/or the amorphous silicon dioxide, has sufficient particle size for pulmonary deposition, even over a lengthy period of time.

For example, the amorphous silicon dioxide can be a hydrophilic silicon dioxide commercially available under the brand name AEROSIL® 200 Pharma or AEROSIL® 300 Pharma. Moreover, the amorphous silicon dioxide can also be a mixture of AEROSIL® 200 Pharma and AEROSIL® 300 Pharma.

The amorphous silicon dioxide may be present in the form of particles, also referred to below as amorphous silicon dioxide particles, preferably in the form of aerosol particles, also referred to below as amorphous silicon dioxide aerosol particles. The amorphous silicon dioxide particles, in particular amorphous silicon dioxide aerosol particles, are preferably not in agglomerated form. In other words, it is preferred that the amorphous silicon dioxide particles, in particular the amorphous silicon dioxide aerosol particles, do not form any agglomerates. Alternatively, it can be preferable that the amorphous silicon dioxide particles, in particular the amorphous silicon dioxide aerosol particles, do not agglomerate, i.e., form no agglomerates, at least for a period of 6 months to 24 months, and in particular 12 months to 24 months.

The amorphous silicon dioxide, in particular the amorphous silicon dioxide particles or amorphous silicon dioxide aerosol particles, may have a mean particle diameter ≤20 μm, in particular 0.1 μm to 18 μm, preferably ≤10 μm, more preferably 0.1 μm to 6 μm, particularly preferably 0.1 μm to 5 μm. Particularly preferably, the amorphous silicon dioxide, in particular the amorphous silicon dioxide particles or amorphous silicon dioxide aerosol particles, have a mean particle diameter <5 μm, in particular 0.1 μm to 4 μm, preferably 0.1 μm to 3 μm, more preferably 0.1 μm to 2 μm, and most preferably 0.1 μm to 1 μm. In particular, the mean particle diameters for the amorphous silicon dioxide have been found to be particularly advantageous for stabilizing the mucolytic substance.

The content of the amorphous silicon dioxide, in particular the amorphous silicon dioxide particles or amorphous silicon dioxide aerosol particles, may be 0.1% by weight to 10% by weight, in particular 0.1% by weight to 5% by weight, preferably 0.1% by weight to 4% by weight, more preferably 0.1% by weight to 3% by weight, particularly preferably 0.2% by weight to 2% by weight, and most preferably 0.5% by weight to 2% by weight based on the total weight of the particular substance mixture.

The amorphous silicon dioxide may have a purity ≥99% by weight, and preferably ≥99.9% by weight.

The amorphous silicon dioxide may have a specific surface area, in particular a BET area (according to DIN-ISO 9277 or DIN-66131), of 50 m²/g to 450 m²/g, in particular 70 m²/g to 400 m²/g, preferably 100 m²/g to 280 m²/g, more preferably 150 m²/g to 350 m²/g, and particularly preferably 170 m²/g to 330 m²/g.

The amorphous silicon dioxide may be present in the form of a powder, in particular in the form of a ground or atomized, preferably micronized powder.

Particularly preferably, the amorphous silicon dioxide is present in the form of a dry powder, in particular in the form of a ground or atomized, preferably micronized dry powder.

In general, any biocompatible or medically compatible substance is suitable as a mucolytic substance, provided that the substance has mucolytic properties within the defined meaning.

The mucolytic substance may be a substance that is grindable or atomizable, in particular micronizable, as a powder, preferably a dry powder.

The substance may be a bronchial mucolytic substance or an expectorant, i.e., a substance that promotes the expectoration or coughing up of bronchial mucus.

The substance may be a secretolytically active substance.

The substance may be a mucolytically active substance.

The substance may be a secretomotorically active substance.

The mucolytic substance may be an organic substance. The organic substance may be an organic molecular compound, a mixture of different organic molecular compounds, an organic salt or an organic salt mixture, i.e., a mixture of different organic salts.

For example, the mucolytic substance can be selected from the group composed of emetine, saponins, acetylcysteine, bromhexine, ambroxol, clenbuterol and mixtures of at least two of the organic compounds.

Alternatively, the mucolytic substance is an inorganic substance. The inorganic substance may be an inorganic molecular compound, a mixture of different inorganic molecular compounds, an inorganic salt or an inorganic salt mixture, i.e., a mixture of different inorganic salts.

Preferably, the mucolytic substance is an inorganic salt or an inorganic salt mixture.

Preferably, the mucolytic substance, in particular the inorganic salt, is an inorganic alkali metal salt, an inorganic alkali metal salt mixture, an inorganic alkaline earth metal salt, an inorganic alkaline earth metal salt mixture or a mixture of at least two of the salts or salt mixtures.

Further preferably, the mucolytic substance, in particular the inorganic salt, is selected from the group composed of inorganic sodium salt, inorganic potassium salt, inorganic magnesium salt, inorganic calcium salt and mixtures of at least two of the salts.

Particularly preferably, the mucolytic substance, in particular the inorganic salt, is selected from the group composed of sodium chloride (NaCl), sodium sulfate (NaSO₄), sodium hydrogen carbonate (NaHCO₃), magnesium chloride (MgCl₂), magnesium sulfate (MgSO₄), calcium chloride (CaCl₂)), calcium sulfate (CaSO₄), ammonium chloride (NH₄Cl) and mixtures of at least two of the salts.

The mucolytic substance, in particular the inorganic salt, may be sodium chloride or a sodium-chloride-containing salt mixture such as, e.g., a mixture of sodium chloride and magnesium chloride.

The mucolytic substance may be in the form of particles, also referred to below as mucolytic substance particles, preferably in the form of aerosol particles, also referred to below as mucolytic substance aerosol particles. The mucolytic substance particles, in particular the mucolytic substance aerosol particles, are preferably not in agglomerated form. In other words, it is preferred if the mucolytic substance particles, in particular the mucolytic substance aerosol particles, form no agglomerates. Alternatively, it can be preferred if the mucolytic substance particles, in particular the mucolytic substance aerosol particles, do not agglomerate, i.e., do not form any agglomerates, at least during a period of 6 months to 24 months, in particular 12 months to 24 months.

The mucolytic substance, in particular the mucolytic substance particles or mucolytic substance aerosol particles, may have a mean particle diameter ≤20 μm, in particular 0.1 μm to 18 μm, preferably ≤10 μm, more preferably 0.1 μm to 6 μm, and particularly preferably 0.1 μm to 5 μm. In particular, the mean particle diameters allow favorable pulmonary deposition without exhibiting unwanted adverse effects.

Particularly preferably, the mucolytic substance, in particular the mucolytic substance particles or mucolytic substance aerosol particles, have a mean particle diameter ≤5 μm. We demonstrated in particular that mucolytic substances having such a particle size (substance particles ground into fine power) allow particularly favorable deposition and thus result in significantly improved treatment and prophylaxis of respiratory disorders. In particular, we demonstrated increased sputum production in the patients treated. In contrast, adverse effects such as pulmonary obstructions, the urge to cough, or taste intolerance were not observed. Preferably, the mucolytic substance, in particular the mucolytic substance particles or mucolytic substance aerosol particles, have a mean particle diameter of 0.1 μm to 4 μm, in particular 0.1 μm to 3 μm, preferably 0.1 μm to 2 μm, and particularly preferably 0.1 μm to 1 μm.

The amorphous silicon dioxide particles, in particular the amorphous silicon dioxide aerosol particles, and the mucolytic substance particles, in particular the mucolytic substance aerosol particles, may be respectively present as independently formed particles, in particular aerosol particles. It is particularly preferred for the amorphous silicon dioxide particles, in particular the amorphous silicon dioxide aerosol particles, and the mucolytic substance particles, in particular the mucolytic substance aerosol particles, not to be connected to one another, in particular neither agglomerated, glued, or otherwise cohesively bonded with one another. It is further particularly preferred for the amorphous silicon dioxide particles, in particular the amorphous silicon dioxide aerosol particles, and the mucolytic substance particles, in particular the mucolytic substance aerosol particles, not to mutually coat one another.

The content of the mucolytic substance, in particular the mucolytic substance particles or mucolytic substance aerosol particles, may be 90% by weight to 99.9% by weight, in particular 95% by weight to 99.5% by weight, preferably 96% by weight to 99% by weight, more preferably 96.5% by weight to 98.5% by weight, and most preferably 97% by weight to 98% by weight based on the total weight of the particular substance mixture.

The mucolytic substance may have a purity ≥99% by weight, and preferably ≥99.9% by weight.

We found that the purity figures are particularly suitable for the prophylaxis and/or treatment of respiratory disorders and yield particularly favorable results in the patients treated. These purity figures were ensured by correspondingly selecting the mucolytic substance to be used. For example, if the mucolytic substance is a chloride salt, salts (so-called medicinal salts) produced by the firm Sanal Salt, AkzoNobel, Wuppertal, Germany, sold under the brand name Sanal® SQ, are suitable for use.

The mucolytic substance may be in the form of a powder, in particular in the form of a ground or atomized, preferably micronized powder.

Particularly preferably, the mucolytic substance is in the form of a dry powder, in particular in the form of a ground or atomized, preferably micronized dry powder.

The particulate substance mixture may further comprise a filler. The filler can be selected in particular from the group composed of mannitol, lactol, lactide, talc and mixtures of at least two of the fillers.

The filler may be in the form of particles, also referred to below as filler particles, preferably in the form of aerosol particles, also referred to below as filler aerosol particles.

The filler, in particular the filler particles or filler aerosol particles, may have a mean particle diameter ≤20 μm, in particular 0.1 μm to 18 μm, preferably ≤10 μm, more preferably 0.1 μm to 6 μm, and particularly preferably 0.1 μm to 5 μm. Particularly preferably, the filler, in particular the filler particles or filler aerosol particles, have a mean particle diameter <5 μm, in particular 0.1 μm to 4 μm, preferably 0.1 μm to 3 μm, more preferably 0.1 μm to 2 μm, and most preferably 0.1 μm to 1 μm.

The filler may be in the form of a powder, in particular in the form of a ground or atomized, preferably micronized powder.

Particularly preferably, the filler is in the form of a dry powder, in particular in the form of a ground or atomized, preferably micronized dry powder.

As mentioned above, the substance mixture is preferably a particulate substance mixture for use in the prophylaxis and/or treatment of a respiratory disorder.

The respiratory disorder can generally be a respiratory disorder or disease in a human or a non-human mammal. Preferably, the respiratory disorder within our meaning is a human respiratory disorder or disease.

The respiratory disorder can in particular be a chronic respiratory disease.

Preferably, the respiratory disorder is a pulmonary disease, in particular chronic pulmonary disease.

Further preferably, the respiratory disorder is selected from the group composed of chronic bronchitis, chronic obstructive bronchitis, chronic obstructive pulmonary disease (COPD), bronchial asthma, bronchiectasis, pulmonary fibrosis such as in particular cystic fibrosis (CF), primary ciliary dyskinesia (PCD) and pulmonary emphysema.

Particularly preferably, the respiratory disorder is chronic bronchitis, chronic obstructive bronchitis, chronic obstructive pulmonary disease (COPD) or cystic fibrosis (CF).

The particulate substance mixture may be prepared for administration by inhalation, preferably dry inhalation. In other words, the particulate substance mixture may be used for administration by inhalation, preferably dry inhalation.

The particulate substance mixture can generally be prepared or used for nasal or oral administration. It is particularly preferable for the particulate substance mixture to be prepared for oral administration or used for oral administration.

The particulate substance mixture may be prepared for capsule inhalation. In other words, the particulate substance mixture is used according to a particularly preferred example for capsule inhalation.

The particulate substance mixture may be in the form of a powder, in particular in the form of a ground or atomized, preferably micronized powder.

Particularly preferably, the particulate substance mixture is in the form of a dry powder, in particular in the form of a ground or atomized, preferably micronized dry powder.

Preferably, the particulate substance mixture is a medicinal powder, i.e., a powder usable in the field of medicine, in particular for use in the prophylaxis and/or treatment of a disease, preferably a dry powder.

The powdered substance mixture may have a mean particle diameter ≤20 μm, in particular 0.1 μm to 18 μm, preferably ≤10 μm, more preferably 0.1 μm to 6 μm, and particularly preferably 0.1 μm to 5 μm. Particularly preferably, the powdered substance mixture has a mean particle diameter <5 μm, in particular 0.1 μm to 4 μm, preferably 0.1 μm to 3 μm, more preferably 0.1 μm to 2 μm, and most preferably 0.1 μm to 1 μm.

The particulate substance mixture may be anhydrous.

The particulate substance mixture may have a water content ≤1% by weight, in particular ≤0.4% by weight, preferably ≤0.3% by weight, more preferably ≤0.2% by weight, and most preferably ≤0.1% by weight based on the total weight of the particulate substance mixture.

The particulate substance mixture may be present in a dosing unit in an amount of 1 mg to 1,000 mg, and in particular either 500 mg to 1000 mg or in particular 100 mg to 500 mg, and more preferably 100 mg to 200 mg. Also preferred are dosing units of 50 mg to 100 mg.

We found that the amounts indicated are those having proven to be suitable as dosing units and in particular are controlled for administration by inhalation, preferably dry inhalation.

The dosing unit may be a capsule. This is advantageous in that the particulate substance mixture can be provided in the desired amount and concentration. This allows optimal and simplified handling, and moreover ensures that no contamination can take place. In addition, the use of a dosing unit is advantageous in that the risk of water absorption by the mucolytic substance can be further reduced.

In general, any hard or soft capsule usable for the formulation of particulate substance mixtures is suitable as a capsule. As a rule, hard and soft capsules are essentially composed of gelatin or other materials such as, e.g., hydroxypropylmethylcellulose (HPMC). Hard capsules ordinarily comprise two mutually closed cylinders hemispherically sealed at one end. For example, such capsules can be filled on a large scale with the mucolytic substance and the amorphous silicon dioxide in the desired amount and concentration while meeting corresponding hygienic and atmospheric conditions. The capsules can be administered using conventional inhalation devices such as, e.g., the so-called Osmohaler® RS-01 Plastiape®. We found that the clinical tolerance and acceptance of such a capsule preparation by the patients were optimal.

The particulate substance mixture may be composed of the mucolytic substance and the amorphous silicon dioxide and optionally an additive such as in particular a filler. With respect to further features and advantages of the substance mixture, in particular the mucolytic substance and the amorphous silicon dioxide and an optional additionally present additive, in particular a filler, reference is made to the above description in its entirety.

The particulate substance mixture may be in the form of a medicinal product or drug or a pharmaceutical formulation. In other words, the particulate substance mixture may be a medicinal product or drug or a pharmaceutical formulation.

We also provide a medicinal product, preferably for use in the prophylaxis and/or treatment of a respiratory disorder.

The medicinal product is characterized in particular by comprising a particulate substance mixture or being composed of such a particulate substance mixture.

Preferably, the medicinal product is prepared for administration by inhalation, preferably dry inhalation. In other words, preferably, the medicinal product is used for administration by inhalation, preferably dry inhalation.

Preferably, the medicinal product is prepared for capsule inhalation. In other words, particularly preferably, the medicinal product is used for capsule inhalation.

In general, the medicinal product can be prepared or used for nasal or oral administration. It is particularly preferable to prepare the medicinal product for oral administration or use it for oral administration.

The medicinal product may be in the form of a powder, in particular in the form of a ground or atomized, preferably micronized powder.

Particularly preferably, the medicinal product is in the form of a dry powder, in particular in the form of a ground or atomized, preferably micronized dry powder.

As mentioned above, the medicinal product is preferably a medicinal product for use in the prophylaxis and/or treatment of a respiratory disorder.

In general, the respiratory disorder can be a respiratory disorder or disease in a human or a non-human mammal. Preferably, the respiratory disorder is a human respiratory disorder or disease.

In particular, the respiratory disorder can be a chronic respiratory disease.

Preferably, the respiratory disorder is a pulmonary disease, in particular a chronic pulmonary disease.

Further preferably, the respiratory disorder is selected from the group composed of chronic bronchitis, chronic obstructive bronchitis, chronic obstructive pulmonary disease (COPD), bronchial asthma, bronchiectasis, pulmonary fibrosis such as in particular cystic fibrosis (CF), primary ciliary dyskinesia (PCD) and pulmonary emphysema.

Particularly preferably, the respiratory disorder is chronic bronchitis, chronic obstructive bronchitis, chronic obstructive pulmonary disease (COPD) or cystic fibrosis (CF).

With respect to further features and advantages of the medicinal product, in particular the particulate substance mixture, to avoid repetition, reference is made to the explanations given in the context of the particulate substance mixture in their entirety. The explanations given therein with respect to the particulate substance mixture also apply (mutatis mutandis) to the medicinal product.

We further provide a dosing unit comprising a particulate substance mixture or a medicinal product.

With respect to further features and advantages of the dosing unit, in particular the particulate substance mixture and the medicinal product, to avoid repetition, reference is made to the explanations given in the context of the particulate substance mixture and the medicinal product in their entirety. The explanations given therein with respect to the particulate substance mixture and the medicinal product also apply (mutatis mutandis) to the dosing unit.

We still further provide a device that administers a particulate substance mixture, a medicinal product or a dosing unit.

The device is characterized in particular by comprising a particulate substance mixture, a medicinal product, or a dosing unit.

The device is preferably an inhalation device, in particular a dry inhalation device.

The device preferably comprises the particulate substance mixture or the medicinal product formulated in a capsule.

For example, a suitable device is the above-mentioned Osmohaler®.

With respect to further features and advantages of the device, in particular the particulate substance mixture, the medicinal product and the dosing unit, to avoid repetition, reference is made to the explanations given in the context of the particulate substance mixture, the medicinal product and the dosing unit in their entirety. The explanations given therein with respect to the particulate substance mixture, the medicinal product and the dosing unit also apply (mutatis mutandis) to the device.

Finally, we provide a method of producing a particulate substance mixture, or a medicinal product.

The method comprises the following steps:

a) providing a particulate, preferably powdered, substance mixture comprising a mucolytic substance and amorphous silicon dioxide, and
b) grinding or atomizing, preferably micronizing, the particulate substance mixture.

This disclosure further based on the surprising finding that upon addition of amorphous silicon dioxide particles to mucolytic substance particles before carrying out a micronizing process, an agglomeration or clumping of the mucolytic substance particles can be prevented, or at least significantly delayed.

Step b) may be carried out by spray drying or a milling gas, in particular in an air-jet mill, preferably in a counter-jet mill.

After step b) is carried out, the substance mixture may be transferred to a dosing unit, and preferably encapsulated.

With respect to further features and advantages of the method, in particular the particulate substance mixture, the mucolytic substance and the amorphous silicon dioxide, to avoid unnecessary repetition, reference is made to the explanations given in the context of the above description, also in their entirety. The examples described therein with respect to the particulate substance mixture, the mucolytic substance and the amorphous silicon dioxide also apply (mutatis mutandis) to the method.

It is understood that the features mentioned above and to be explained below can be implemented not only in the respectively indicated combination, but also in other combinations or individually, without departing from the scope of this disclosure.

We now provide explanations by examples, from which further properties and advantages can be derived. The examples are given solely as examples and do not limit the scope of this disclosure.

### EXAMPLES

1. Production of a Dry Powder from Micronized Sodium Chloride and Amorphous, Hydrophilic Silicon Dioxide

Taking Sanal® SQ sodium chloride (Akzo Nobel) and amorphous, hydrophilic silicon dioxide (AEROSIL® 200 Pharma, Evonik Industries) as starting materials, aerosol particles with a mean particle diameter <5 μm were produced using a Micronizer SaltPro 3 from the firm Microsalt International BV according to the manufacturer's instructions.

The salt and silicon grains were comminuted to the desired size in the Micronizer by cyclone technology, forming an aerosol. This cyclone produced a specified speed at which the salt and silicon grains rotated. Based on the size of the turbulence chamber, the chamber was first filled with a small amount of salt and silicon grains before the cyclone was started. This minimum filling was necessary to achieve an optimum concentration of salt and silicon dioxide in the cyclone. An excessively low or excessively high density resulted in insufficient particle quality. As soon as the cycle started, the particles were mechanically comminuted. As soon as the particles had reached the desired size, they were precipitated by the negative pressure in the cyclone chamber and transported out of the Micronizer. Excessively large particles remained in the cyclone until their decreasing weight allowed precipitation by negative pressure.

It was possible to adjust the size of the salt and silicon dioxide particles by varying the negative pressure. The cyclone speed and the length of the Micronizer tube were further decisive parameters for aerosol quality. The amount of particles released with the desired size was approximately 1 g/min. During this process, moreover, 1 g of salt and silicon dioxide per minute was actively transported by a dosing system into the cyclone chamber to continuously ensure the desired density and thus the quality of the aerosol. The Micronizer SaltPro 3K was capable of continuously producing aerosol, with the release of approximately 1 g/min being ensured.

### 2. Formulation of the Micronized Powder Mixture in a Hard Capsule

Size 3 two-piece hard gelatin capsules from the firm CAPSUGEL®, Peapack, USA were used. The capsules were filled with 50 mg or 100 mg and sealed.

### 3. Clinical Trial

The therapeutic efficacy of halotherapy was investigated on five consecutive days for periods of 45 minutes each in six volunteers with CF. The trial was carried out at the facilities of the Sports and Swimming Center Fildorado GmbH, Filderstadt, Germany. Secretolysis was tested based on the amount of sputum, possibly increased elimination of leukocytes and Pseudomonas bacteria in the sputum, and the pulmonary function parameters FVC, FEV1 and MMEF before beginning the 45-minute therapy, immediately thereafter, and one hour thereafter. Adverse effects were observed as further target parameters. In contrast to hypertonic sodium chloride therapy, no anti-obstructive agent such as a β-sympathomimetic was administered at the beginning of halotherapy. During treatment, the patients were continuously monitored, both clinically and by pulse oximetry.

The treatment was carried out in parallel in 15 volunteers with CF. Each patient was administered anhydrous medicinal sodium chloride (Sanal® SQ) and amorphous hydrophilic silicon dioxide (Aerosil® 200 Pharma, Evonik Industries) respectively in the form of aerosol particles with a mean particle diameter ≤1 μm. For this purpose, the powder mixture was provided by the University Pharmacy, Tübingen in hard capsules (50 mg per capsule). On a trial basis, 15 adult volunteers were allowed to inhale two capsules each using an Osmohaler (Osmohaler®, RS-01 Plastiape®, Italy). These patients were also not given premedication with β-sympathomimetics.

Our capsule preparation was found by the patients to be clinically tolerable and acceptable.

In the halotherapy conducted as a reference, an increase in sputum production during and after the 45-minute treatment period and a significant reduction in leukocyte count and the plate count of Pseudomonas aeruginosa in the sputum were observed compared to baseline. There was also an improvement in the pulmonary function parameters FVC, FEV1 and MMEF after five days.

In comparison, the 15 patients treated according to our protocols first showed problem-free tolerance of inhalation of a total of 100 mg of the administered anhydrous powder mixture (sodium chloride and amorphous hydrophilic silicon dioxide). The patients were clinically and spirometrically examined before and after inhalation. Adverse effects such as pulmonary obstruction, and increased urge to cough, or taste intolerance were not observed. All patients showed significantly increased sputum production.

We were thus able to impressively demonstrate that the use of mucolytic substance particles in combination with amorphous silicon dioxide particles is outstandingly well-suited for the prophylaxis and/or treatment of respiratory disorders.

1-20. (canceled)

21. A method for preventing and/or treating a respiratory disorder, comprising the step of administering a particulate substance mixture to a human or non-human mammal, wherein the particulate substance mixture comprises a mucolytic substance and amorphous silicon dioxide, wherein the amorphous silicon dioxide is present in the form of particles having a mean particle diameter <20 μm and wherein the mucolytic substance is present in the form of particles having a mean particle diameter <20 μm.

22. The method as claimed in claim 21, wherein the amorphous silicon dioxide is hydrophilic silicon dioxide.

23. (canceled)

24. The method as claimed in claim 21, wherein an amount of the amorphous silicon dioxide is 0.1% by weight to 10% by weight, based on the total weight of the particulate substance mixture.

25. The method as claimed in claim 21, wherein the mucolytic substance is a secretolytically, mucolytically or secretomotorically active substance.

26. The method as claimed in claim 21, wherein the mucolytic substance is an organic compound selected from the group consisting of emetine, saponins, acetylcysteine, bromhexine, ambroxol, clenbuterol and mixtures of at least two of said organic compounds.

27. The method as claimed in claim 21, wherein the mucolytic substance is an inorganic salt.

28. The method as claimed in claim 21, wherein the mucolytic substance is selected from the group consisting of sodium chloride, magnesium chloride, magnesium sulfate, calcium chloride, calcium sulfate, ammonium chloride and mixtures of at least two of said salts.

29. (canceled)

30. The method as claimed in claim 21, wherein content of the mucolytic substance is 90% by weight to 99.9% by weight, based on the total weight of the particulate substance mixture.

31. The method as claimed in claim 21, wherein the particulate substance mixture further comprises a filler selected from the group consisting of mannitol, lactol, lactide, talc and mixtures of at least two of said fillers.

32. The method as claimed in claim 21, wherein the respiratory disorder is a pulmonary disease selected from the group consisting of chronic pulmonary disease, chronic bronchitis, chronic obstructive bronchitis, chronic obstructive pulmonary disease (COPD), bronchial asthma, bronchiectasis, pulmonary fibrosis, cystic fibrosis (CF), primary ciliary dyskinesia (PCD) and pulmonary emphysema.

33. The method as claimed in claim 21, wherein the substance mixture is prepared or used for administration by inhalation or dry inhalation.

34. The method as claimed in claim 21, wherein the substance mixture is prepared or used for oral administration.

35. The method as claimed in claim 21, wherein the substance mixture is present in a dosing unit in particular in an amount of 1 mg to 1000 mg.

36. The method as claimed in claim 21, wherein the substance mixture is present in the form of at least one of a powder, ground powder, micronized powder, and micronized dry powder.

37-39. (canceled)
